(19) United States
(12) Patent Application Publication
(10) Pub. No.: US 2010/0303937 A1
(43) Pub. Date: Dec. 2, 2010

(54) NOVEL COMPOSITION TO INCREASE TESTOSTERONE LEVELS

(76) Inventor: Michael FARBER, Montreal (CA)

Correspondence Address: KLAUBER & JACKSON, 411 HACKENSACK AVENUE, HACKENSACK, NJ, 07601, US

(21) Appl. No.: 12/790,947

(22) Filed: May 31, 2010

Related U.S. Application Data
(60) Provisional application No. 61/182,873, filed on Jun. 1, 2009.
(60) Provisional application No. 61/227,993, filed on Jul. 23, 2009.
(60) Provisional application No. 61/234,861, filed on Aug. 18, 2009.
(60) Provisional application No. 61/227,238, filed on Jul. 21, 2009.

(51) Int. Cl. A61K 36/48; A61P 5/24

(52) U.S. Cl. 424/757

(57) ABSTRACT

The present invention relates to a composition for increasing testosterone physiological levels comprising: a) a sufficient amount of at least one aromatase inhibitor chosen from a flavone substituted with at least one methoxy group at position 3′,4′,5 or 7, any combinations thereof, and any di, tri or tetra combinations thereof; and a flavanone substituted with at least one methoxy group at position 3′,4′,5 or 7, any combinations thereof, and any di, tri or tetra combinations thereof; and b) a sufficient amount of at least one 5α-reductase inhibitor that inhibit testosterone conversion to DHT, such as beta-sitosterols; in association with a pharmaceutically acceptable carrier.

### CROSS REFERENCE TO RELATED APPLICATIONS

The present application claims the priority of the following co-pending provisional applications: U.S. Ser. No. 61/182,873, filed on Jun. 1, 2009; U.S. Ser. No. 61/227,238, filed Jul. 21, 2009; U.S. Ser. No. 61/227,993, filed Jul. 23, 2009; and U.S. Ser. No. 61/234,861, filed Aug. 18, 2009. Applicant claims the benefits of all such applications under 35 U.S.C. §119(e), and the disclosures of all applications are incorporated herein by reference in their entireties.

### BACKGROUND OF THE INVENTION

(a) Field of the Invention

The present invention relates to compositions and corresponding methods for increasing testosterone physiological levels, and particularly, to such compositions that may include an aromatase inhibitor.

(b) Description of Prior Art

Testosterone is the principal male androgen and is responsible for the development and maintenance of male sexual characteristics, including external virilization, sexual maturity at puberty, spermatogenesis, sexual behaviour, libido and erectile functioning. It also supports bone and muscle tissue development during growth. However, after physical maturity, the level of circulating testosterone starts to decline, possibly leading to a diminution in muscle mass. Therefore, there is a growing need for the development of some form of androgen replacement for the elimination of the effects encountered on muscle mass, which is a specific concern for men evolving in the sports and bodybuilding domains.

Several methods for re-establishing androgen levels to their pre-adult concentrations in men were developed with injectable preparations. U.S. Pat. No. 6,989,378, between others, relates to a novel androgen, (7α, 17β)-7-methyl-17-[(1-ox-oundecyl)oxy]estr-4en-3one (MENT undecanoate), having a good solubility in oily media and being particularly suitable for administration by means of injection. Injectable medias are normally fashioned in order to allow slow and sustained hormone release in the blood of the patient over various preset periods of time. However, the main problem with such inventions is that they usually end up providing inconsistent dosing because of a great variance in hormone release between the site of injection and the rest of the body. Moreover, injection of testosterone preparations usually entails very high concentrations from the moment of the administration followed by a period of subnormal hormone concentrations. Because of such uncontrolled release of the active agent, various side-effects developed in periods of high hormone concentrations, such as gynecomastia, water retention, edema and increased fat deposition.

Some methods of treatment for restoring the testosterone concentrations in adult males with declining levels focused on the administration of a metabolic precursor of testosterone. U.S. Pat. No. 5,880,117 relates to a method of administering the testosterone precursor 4-androstene-3β, 17β-diol as a means of increasing testosterone levels in humans. The invention proposes a compound which concentration peaks within 90 minutes of its administration and declines thereafter over a period of 3 to 4 hours. Even if the androgen preparation has shown easy conversion to testosterone in the physiologic environment, it still lacks constant repartition in the organs of predilection and can possibly entail various side-effects. U.S. Pat. No. 6,451,782 is based on the administration of 4-androstene-3α, 17β-diol, a direct precursor hormone to testosterone, in order to increase testosterone levels in male subjects. However, even if conversion to testosterone has been demonstrated as being much more complete than in other cases, the release kinetic of the compound were still not ideal.

Other proposed methods of treating the present condition were related to the physiologic administration of synthetic androgen derivatives of testosterone such as methyltestosterone, fluoxymesterone and stanozol. Those compounds were alkylated at the 17^{th} carbon in order to restrain any reduction of the metabolite to its inactive from. Such innovation induced an increase in the bioavailability of the compound, which allowed a more constant release of the active agent in the physiologic environment. However, patients encountered possible risks of developing complications with liver functions, which highly diminishes the convenience of using such technology.

Steroidal based aromatase inhibitors, androsta-1,4,6-triene-3, 17-dione (ATD) specifically, have been cited in the literature on numerous occasions over the past thirty years. It was first used to elucidate the nature of the enzyme aromatizing androstenedione and testosterone. The effects of aromatase inhibition upon sex steroids in men (in this case older eugonadal men) were definitively and quantitatively studied wherein it was shown that administration of an aromatase inhibitor to 15 men over 65 caused significant decreases in estrogen and its related compounds and significantly increased testosterone.

It is well known that various flavones and flavanones can inhibit aromatase activity, such as chrysin and quercetin among others. The biggest problem with the administration of flavones and flavanones is their poor absorption characteristics due to enzymatic degradation in the intestine and poor absorption across the intestinal epithelium.

It would therefore be highly desirable to be provided with a potent fast acting aromatase inhibitor displaying only slight or negligible binding to the peripheral androgen receptors that would rapidly raise testosterone levels in male subjects, have a half life allowing for at most twice daily ingestion and have sufficient binding to the hypothalamic androgen receptor sites to downregulate the feedback loop.

### SUMMARY OF THE INVENTION

In accordance with a first embodiment of the present invention, there is provided a composition for increasing testosterone physiological levels comprising: a) a sufficient amount of at least one aromatase inhibitor chosen from a flavone substituted with at least one methoxy group at position 3′,4′,5 or 7, any combinations thereof, and any di, tri or tetra combinations thereof; and a flavanone substituted with at least one methoxy group at position 3′,4′,5 or 7, any combinations thereof, and any di, tri or tetra combinations thereof; and b) a sufficient amount of at least one 5α-reductase inhibitor; in association with a pharmaceutically acceptable carrier.

Such an inhibitor of 5α-reductase may be chosen from

sterols;

lignans;

enterolactones;

β-sitosterone;

coumarins;

ginsenosides; and

acetonides.

The composition may further comprise sterols and other compounds that inhibit testosterone conversion to DHT, such as lignans, and other compounds that inhibit binding of testosterone to SBGH (sex binding globulin hormone), agents whether natural or other that inhibit the action of aromatase enzymes on testosterone, coumarins, ginsenosides, acetonides, agents that augment testosterone secretion such as gonadins, tripeptide Glu-Asp-Pro amide, saponins, or cordycep sinensis extract, aqueous extract of Zingiber officianale and any combination thereof. The composition can be orally, parentally or transdermally administered. The preferred route is oral administration and the most preferred route is oral administration of an enteric coated PEG carrier.

In accordance with a further embodiment of the present invention, the composition further comprises a sufficient amount of at least one of:

c) an agent stimulating production of endogenous testosterone;
d) an agent stimulating production of endogenous luteinizing hormone; and
e) an agent preventing binding of testosterone to SBGH.

The agent stimulating production of endogenous testosterone in c) above is chosen from:

gonadins;

tripeptide Glu-Asp-Pro amide;

cordycep sinensis extract;

aqueous extract of Zingiber officianale; and

any combination thereof.

The agent stimulating production of endogenous luteinizing hormone in d) above is chosen from:

saponins;

avena sativa extract; and

any combination thereof.

The agent preventing binding of testosterone to SBGH (sex binding globulin hormone) in e) above is chosen from:

3,4-divan illyltetrahydrofuran.

The composition can be orally, parentally or transdermally administered. The preferred route is oral administration and the most preferred route is oral administration of an enteric coated PEG carrier.

In accordance with a preferred embodiment of the present invention, the flavones, flavanones, sterols, lignans, enterolactones, saponins, coumarins, and other agents, are natural or synthetic.

In accordance with a preferred embodiment of the present invention, the composition comprises

7 methoxyflavone or 4′,7-dimethoxyflavone, and

Beta-sitosterols, in association with a PEG/400 water carrier in an enteric PEG capsule.

In accordance with a preferred embodiment of the present invention, the composition comprises

7 methoxyflavone or 4′,7-dimethoxyflavone;

Beta-sitosterol;

3,4-divanillyl tetrahydrofuran;

Cordyceps sinensis;

6,7 dihydroxubergamottin (DHB); and

EGCG or epicatechin, in association with a PEG/400 water carrier in an enteric PEG capsule.

Preferably, the composition comprises two layers of said 7 methoxyflavone and said Beta-sitosterols for delivery in two bursts.

Preferably, the composition comprises between 10 mg to 100 mg of 7 methoxyflavone, and between 10 mg and 1000 mg of Beta-sitosterols, and between 10 mg and 200 mg of 3,4-divanillyl tetrahydrofuran.

In accordance with a preferred embodiment of the present invention, the composition comprises

7 methoxyflavone; and

Beta-sitosterols, in association with an oral enteric coated PEG microcapsule for oral delivery.

Preferably, the oral composition comprises two layers of said 7 methoxyflavone and said Beta-sitosterols for delivery in two bursts.

Preferably, the oral composition comprises between 0.25 mg to 15 mg of tripeptide Glu-Asp-Pro amide or between 25 mg to 250 mg of a cordycep sinensis extract.

Preferably, the oral composition comprises between 10 mg to 100 mg of 7 methoxyflavone, and between 100 mg and 1000 mg of Beta-sitosterols, and between 10 mg and 200 mg of 3,4-divanillyl tetrahydrofuran.

Preferably, the oral composition further comprises agents to increase growth hormone level and/or thyroid activity.

In accordance with another preferred embodiment of the present invention, there is provided a method for increasing testosterone physiological levels in a male subject, which comprises administering a sufficient amount of the composition of the present invention.

In accordance with another preferred embodiment of the present invention, the administration of the composition is transdermal or intranasal.

In accordance with another preferred embodiment of the present invention, the administration of the composition is oral.

In accordance with another preferred embodiment of the present invention, the oral composition further comprises agents to increase growth hormone level and/or thyroid activity.

In accordance with another preferred embodiment of the present invention, the daily total dosage of the composition to administer is of about 50 to 2000 mg.

In accordance with another preferred embodiment of the present invention, the daily total dosage is administered at least two times a day.

In accordance with another preferred embodiment of the present invention, the daily total dosage is divided in two equal dosages to be administered at twelve hours intervals.

In accordance with another preferred embodiment of the present invention, the daily total dosage is divided in three equal dosages to be administered at eight hours intervals.

In accordance with another preferred embodiment of the present invention, the testosterone level is significantly increased.

In accordance with another preferred embodiment of the present invention, there is provided a method for improving muscle strength, athletic performances and/or lean body mass gain, which comprises administering a sufficient amount of the composition of the present invention.

In accordance with another preferred embodiment of the present invention, the administration of the composition is transdermal or intranasal.

In accordance with another preferred embodiment of the present invention, the administration of the composition oral.

In accordance with another preferred embodiment of the present invention, the daily total dosage of the composition to administer is of about 25 to 1000 mg.

In accordance with another preferred embodiment of the present invention, the daily total dosage is administered at least two times a day.

In accordance with another preferred embodiment of the present invention, the daily total dosage is divided in two equal dosages to be administered at twelve hours intervals.

In accordance with another preferred embodiment of the present invention, the daily total dosage is divided in three equal dosages to be administered at eight hours intervals.

In accordance with still another preferred embodiment of the present invention, muscle size is increased.

All references referred herein are hereby incorporated by reference.

For the purpose of the present invention, the following terms are defined below.

The term “polyethylene glycol” or PEG is intended to mean an oligomer or polymer of ethylene oxide. It is also known as poly(ethylene oxide) (PEO) or polyoxyethylene (POE).

The term “enteric coating” is intended to mean a barrier applied to oral medication to control the location in the digestive system where the oral medication released and absorbed. Enteric coatings prevent release of medication before it reaches the small intestine.

The expression “supraphysiological levels” as used herein refers to amounts greater than normally found in the human body.

The expression “significantly increased” refers to a rapid production higher than normal physiological levels of the human body.

Microspheres may also be used as digestible excipients to produce layered sugar-coating structures onto which an active substance of pegylated glutathione and/or other substance(s) are coated in order to allow sustained release of these elements.

### DETAILED DESCRIPTION OF THE INVENTION

The present invention relates to a composition for increasing testosterone physiological levels comprising: a) a sufficient amount of at least one aromatase inhibitor chosen from a flavone substituted with at least one methoxy group at position 3′,4′,5 or 7, any combinations thereof, and any di, tri or tetra combinations thereof; and a flavanone substituted with at least one methoxy group at position 3′,4′,5 or 7, any combinations thereof, and any di, tri or tetra combinations thereof; and b) a sufficient amount of at least one 5α-reductase inhibitor that inhibit testosterone conversion to DHT, such as sterols; in association with a pharmaceutically acceptable carrier.

The composition may further comprise a sufficient amount of at least one:

c) an agent stimulating production of endogenous testosterone;
d) an agent stimulating production of endogenous luteinizing hormone; and
e) an agent preventing binding of testosterone to SBGH.

The composition can be orally, parentally or transdermally administered. The preferred route is oral administration and the most preferred route is oral administration of an enteric coated PEG carrier.

The methoxy substituted flavone and methoxy substituted flavanone used include highly purified extracts of plant origin standardized to 95%,or their synthetic equivalent, beta-sitosterols from soy or other paints standardized to 98% purity, and nettle root extract standardized to greater than 95% for 3,4-divanillyl tetrahydrofuran. These extracts can be used alone or in combination with other agents to promote testosterone secretion or inhibit DHT production or effects.

The flavones and/or flavanones/5 alpha reductase combination may be further combined with extracts such as ginsenosides, saponins and or lignins, enterolactones, coumarins, and other agents which possess anti-aromatase activity.

The purified extracts are then enclosed in appropriate PEG solutions and enteric coated.

Enteric coating can also mean an pH sensitive or plain erosion type coating such as a high molecular weight hydrogenated or partially hydrogenated vegetable oil or derivative mixture that will disintegrate slowly upon ingestion thus protecting the inner core for a period of time (typically 1 to 2 hours in vitro) to allow transit thru the stomach and into the small intestine before beginning disintegration. Enteric coatings are often used to prevent stomach irritation caused by some medicine or to safeguard the medicine or compounds against the stomach acids or enzymes of the gastric environment. Therefore, an enteric coating will help control the location in the digestive system where it is absorbed. Enteric coatings are selectively insoluble substances that will not dissolve in the acidic juices (pH˜3) of the stomach, but upon reaching the relatively higher pH (pH>5.5) environment of the small intestine will readily dissolve. Materials used for enteric coatings include fatty acids, waxes, and shellac as well as plastics (methylacrylates). Suitable materials used for enteric coatings include but are not limited to: methacrylic acid copolymers, cellulose acetate (including succinate and phthalate versions), styrol maleic acid co-polymers, polymethacrylic acid/acrylic acid copolymer, hydroxypropyl methyl cellulose phthalate, polyvinyl acetate phthalate, hydroxyethyl ethyl cellulose phthalate, hydroxypropyl methyl cellulose acetate succinate, cellulose acetate tetrahydrophtalate, acrylic resin, timellitate, shellac, alginic acid, medium chain triglycerides, oleic acid, stearic acid. Hydrogenated and partially hydrogenated vegetable oils and derivatives used as enteric coatings are cottonseed oil, soybean oil, palm oil, palm kernel oil, and other vegetable derived hydrogenated oils with melting points typically in the 55-70° C. range. These vegetable oils are being applied in these systems to provide enteric protection and delayed release of the active components in the bead formulas and dosage forms; and considered to be unique and atypical up to this point in time.

Also, the composition may include minerals. Minerals include potassium iodide, cupric (sulfate anhydrous, picolinate, sulfate monohydrate, trioxide, selenomethionine, borates, zinc, calcium, magnesium, chromium, manganese, molybdenum, and iron.

Microgranules, (also called neutral pellets, nonpareil seeds, sugar spheres or sugar beads) are inert spherical granules often composed of sucrose and/or maize (corn) starch, or other ingredients which are pharmacologically indifferent, digestible excipients, frequently present in the normal diet. Microgranules are often produced using a layered sugar-coating structure. The result is microgranules with sufficient mechanical stability for further processing. The ideally rounded microgranules classed in closely graduated particle sizes are then coated with active substance(s) and/or sustained release additives. The core of the finished pellet contains no active substance itself so that this solution is used for low-dose substances or substances with a high effect/dose relation. But the use of small sugar spheres and corresponding procedures also makes it possible to use this method to produce pellets containing more than 75% active substance. Microgranules may be used as excipients in the capsule and tablet formulation of the present invention, particularly in multiparticulate formulations. They are the core upon which the composition of the present invention are coated, usually used for controlled or sustained release drug delivery technologies. The microgranules may be coated with the composition of the present invention, which are then coated with a film, such as an enteric coating or any other suitable film. Subsequently, a secondary or even a tertiary layer of sugar may be added and coated with the composition of the present invention for release of the active molecules in two or even three bursts.

One important requirement for uniform application of the active substance(s) in subsequent coating is the particle size of the microgranules. Calculating the surface-per-input quantity also depends on a uniform particle size, as well as a spherical shape. The particle size is defined in micrometer (gm) and according to the international sieve series, whereby the partly uneven numbers of the nominal mesh widths come from conversion from the still common ASTM standard sieves.

The uptake of the flavones can also be upgraded by the addition of coumarins to the composition, between 25-5 mg per dose may be used to interfere with binding of the methoxyflavone to CYP enzymes in the Caco 2 layer of the small intestine.

More specifically it is shown that a 5 to 75 mg dose of 7 methoxyflavone along with 5 mg to 75 mg dose of tripeptide and a 10 to 1000 mg dose of β-sitosterone will inhibit aromatase activity and lead to significant rise in testosterone levels.

Another preferred composition comprises 7 methoxyflavone, Beta-sitosterols and 3,4-divanillyl tetrahydrofuran which inhibits testosterone binding to sex hormone binding globulin (SHBG) and increases free testosterone levels, in association with an oral enteric PEG coated microsphere or in the form of a powder dissolved in a PEG/400 water carrier in an enteric PEG capsule or microsphere. The composition may include other agents to raise testosterone levels, to inhibit DHT formation, to raise energy level, to improve anabolic factors, to increase growth hormone levels or increase thyroid activity.

The oral enteric coated delivery system allows for methoxy flavones and flavanones to avoid being metabolized in the stomach and intestine with a possible first pass metabolism in the liver.

The PEG/400 water mixture solubilises the agents such as 3,4-divanillyl tetrahydrofuran and will have increased absorption in the intestine.

Another preferred composition comprises two layers of said 7 methoxyflavone, Beta-sitosterols and said 3,4-divanillyl tetrahydrofuran for delivery in two bursts. The second layer creating a core and preferably being coated with an enteric coating and the first layer surround the core and being coated with an enteric coating to provide a first layer being release first. The second layer or core is being release after complete release of the first layer. Each layer may be slow release or burst release. Preferably, the layers are release in two bursts.

Preferably, the composition comprises between 10 mg to 100 mg of 7 methoxyflavone, and between 100 mg and 1000 mg of Beta-sitosterols and between 10 mg and 200 mg of 3,4-divanillyl tetrahydrofuran.

The oral enteric coated PEG microsphere allows for passage through the gastric environment without degradation. The PEG solution helps to “solubilise” the included compounds whether they are lipophilic or hydrophilic. The PEG solution includes further compounds such as polysorbate 80, decanoic acid and others to enhance intestinal absorption and/or impede degradation by Caco-2 enzymes.

### EXAMPLE 1

### Testing to Prove Increases in Testosterone from 7 Methoxyflavone Composition

This is a double blind study with 20 male subjects aged between 25 to 35. First ten subjects are taking the 7 methoxyflavone composition and second ten subjects are taking a placebo for a period of 7 days. Then 7 days of wash out and then the reverse with the first ten subjects taking the placebo and the second ten subjects taking the 7 methoxyflavone composition.

The 7 methoxyflavone capsules consists of:

25 mg of 7 methoxyflavone;

600 mg beta-sitosterols; and

900 mg fenugreek extract.

Subjects are taking four capsules daily at 8:00pm.

Testosterone readings will be made just prior to ingestion and 12 hours later at 8:00 am the following morning.

Measurements of free testosterone level and total testosterone level are done by saliva testing (ZRT laboratories immunoassay saliva testing).

The objective is to increase total testosterone level by 50% over placebo in 12 hours.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition for increasing testosterone physiological levels comprising:
a) a sufficient amount of at least one aromatase inhibitor chosen from a flavone substituted with at least one methoxy group at position 3′,4′,5 or 7, any combinations thereof, and any di, tri or tetra combinations thereof; and a flavanone substituted with at least one methoxy group at position 3′,4′,5 or 7, any combinations thereof, and any di, tri or tetra combinations thereof; and
b) a sufficient amount of at least one 5α-reductase inhibitor; in association with a pharmaceutically acceptable carrier.

2. The composition according to claim 1, wherein said inhibitor of 5α-reductase is chosen from
sterols;
lignans;
enterolactones;
13-sitosterone;
coumarins;
ginsenosides; and
acetonides.

3. The composition according to claim 2, which further comprises a sufficient amount of at least one of:
c) an agent stimulating production of endogenous testosterone;
d) an agent stimulating production of endogenous luteinizing hormone; and
e) an agent preventing binding of testosterone to SBGH.

4. The composition according to claim 3, wherein said agent in c) is chosen from:
gonadins,
tripeptide Glu-Asp-Pro amide;
cordycep sinensis extract;
aqueous extract of Zingiber officianale; and
any combination thereof.

5. The composition according to claim 3, wherein said agent in d) is chosen from:
saponins;
avena sativa extract, and
any combination thereof.

6. The composition according to claim 3, wherein said agent in e) is chosen from:
3,4-divanillyl tetrahydrofurans.

7. The composition according to claim 1, wherein said pharmaceutically acceptable carrier is an oral enteric coated PEG carrier.

8. The composition according to claim 1, which comprises
7 methoxyflavone or 4′,7-dimethoxyflavone; and
Beta-sitosterols, in association with a PEG/400 water carrier in an enteric PEG capsule.

9. The composition according to claim 1, which comprises
7 methoxyflavone or 4′,7-dimethoxyflavone;
Beta-sitosterols;
3,4-divanillyl tetrahydrofurans;
Cordyceps sinensis,
6,7 dihydroxubergamottin (DHB); and
EGCG or epicatechin, in association with a PEG/400 water carrier in an enteric PEG capsule.

10. The composition according to claim 8, which comprises two layers of said 7 methoxyflavone and said beta-sitosterols for delivery in two bursts.

11. The composition according to claim 8, wherein said composition comprises between 10 mg to 100 mg of 7 methoxyflavone, and between 10 mg and 1000 mg beta-sitosterols and between 10 mg and 200 mg of 3,4-divanillyl tetrahydrofuran.

12. The composition according to claim 1, which comprises
7 methoxyflavone; and
Beta-sitosterols, in association with an enteric coated PEG carrier for oral delivery.

13. The composition according to claim 12, which comprises two layers of said 7 methoxyflavone and said Beta-sitosterols for delivery in two bursts.

14. The composition according to claim 12, which further comprises between 0.25 mg to 15 mg of tripeptide Glu-Asp-Pro amide or between 25 mg to 250 mg of a cordycep sinensis extract.

15. The composition according to claim 12, wherein said composition comprises between 10 mg to 100 mg of 7 methoxyflavone, and between 10 mg and 1000 mg of Beta-sitosterols and between 10 mg and 200 mg of 3,4-divanillyl tetrahydrofuran.

16. The composition of claim 12, further comprising agents to increase growth hormone level and/or thyroid activity.

17. A method for increasing testosterone physiological levels in a male subject, which comprises administering a sufficient amount of the composition of claim 1.

18. The method according to claim 17, wherein said testosterone levels are increased to high physiological or supraphysiological levels.

19. The method according to claim 17, wherein said administration is transdermal or intranasal.

20. The method according to claim 17, wherein said administration is oral.

21. The method according to claim 17, wherein said amount is a daily total dosage of about 50 to 2000 mg.

22. The method according to claim 17, wherein said daily total dosage is administered at least two times a day.

23. The method according to claim 22, wherein said daily total dosage is divided in two equal dosages to be administered at twelve hours intervals.

24. The method according to claim 22, wherein said daily total dosage is divided in three equal dosages to be administered at eight hours intervals.

25. A method for improving a male's libido, muscle strength, athletic performances and/or lean body mass gain, which comprises administering a sufficient amount of the composition of claim 1.

26. The method according to claim 25, wherein said administration is transdermal or intranasal.

27. The method according to claim 25, wherein said administration is oral.

28. The method according to claim 25, wherein said amount is a daily total dosage of about 25 to 1000 mg.

29. The method according to claim 28, wherein said daily total dosage is administered at least two times a day.

30. The method according to claim 29, wherein said daily total dosage is divided in two equal dosages to be administered at twelve hours intervals.

31. The method according to claim 29, wherein said daily total dosage is divided in three equal dosages to be administered at eight hours intervals.

32. The method according to claim 29, wherein muscle size is increased.
